(19) United States
(12) Patent Application Publication
(10) Pub. No.: US 2023/0402143 A1
(43) Pub. Date: Dec. 14, 2023

(54) SYSTEM AND METHOD FOR MONITORING AND MANAGING INDIVIDUAL WELLBEING IN ORGANISATIONS

(71) Applicant: LIMINAL GROUP PTY LTD, INVERLOCH, Victoria (AU)

(72) Inventor: William George Hams, Victoria (AU)

(21) Appl. No.: 18/249,047

(22) PCT Filed: Oct. 15, 2021
(86) PCT No.: PCT/IB2021/059483

§ 371 Date: Apr. 13, 2023

(30) Foreign Application Priority Data
Oct. 15, 2020 (AU) 2020903738

(51) Int. Cl. G16H 20/00; G16H 10/20

(52) U.S. Cl. CPC G16H 20/00; G16H 10/20

(57) ABSTRACT

A system, comprising a memory, and a processor configured by instructions stored in the memory to receive wellbeing data from an individual within an organisation, apply one or more statistical or machine learning models to the wellbeing data of the individual to provide wellbeing recommendations to the individual to support their wellbeing, wellbeing metrics to the organisation and the individual to monitor the individuals wellbeing, intervention triggers to the organisation to manage the individuals wellbeing.

### FIELD

The present invention relates generally to a system and method for monitoring and managing wellbeing of individuals within organisations.

### BACKGROUND

Organisations are increasingly recognising the importance of measuring, monitoring and managing the physical, social, mental, and emotional wellbeing of individuals within their organisation, for example, students within educational institutions, employees within workplaces, athletes within sports clubs, etc.

Organisations traditionally measure individual wellbeing using annual, large-scale wellbeing surveys, followed by pulse surveys to track wellbeing month-to-month or quarter-to-quarter. While such surveys allow baseline and snap-shot measurements of individual wellbeing at an organisational level, they do not enable organisations to monitor and manage wellbeing dynamically and proactively in real time at an individual level.

In view of this background, there is an unmet need for improved solutions for monitoring and managing wellbeing of individuals within organisations.

### SUMMARY

According to the present invention, there is provided a system, comprising:
a memory; and
a processor is configured by instructions stored in the memory to:
   receive wellbeing data from an individual within an organisation;
   apply one or more statistical or machine learning models to the wellbeing data of the individual to provide:
      wellbeing recommendations to the individual to support their wellbeing;
      wellbeing metrics to the organisation and the individual to monitor the individual's wellbeing;
      intervention triggers to the organisation to manage the individual's wellbeing.

The wellbeing data may comprise survey data, daily check-in data, and help-ticket data.

The wellbeing recommendations provided to the individual may comprise recommended actions and recommended digital content to support their wellbeing.

The wellbeing recommendations may be generated by applying the one or more statistical or machine learning models to the survey data of the individual.

The wellbeing metrics provided to the organisation and the individual may comprise wellbeing scores and wellbeing trends of the individual and the organisation.

The wellbeing scores and wellbeing trends of the individual and the organisation may be generated by applying the one or more statistical or machine learning models to the survey data and daily check-in data of the individual.

The wellbeing scores and wellbeing trends of the individual and the organisation may be provided to the organisation and the individual via wellbeing dashboards and wellbeing reports.

The intervention triggers provided to the organisation may be generated by applying the one or more statistical or machine learning models to the survey data, daily check-in data, help-ticket data, and whether or not the individual has actioned the wellbeing recommendations.

The present invention also provides a method, comprising:
receiving, at a processor, wellbeing data from an individual within an organisation;
applying, by the processor, one or more statistical or machine learning models to the wellbeing data of the individual to provide:
   wellbeing recommendations to the individual to support their wellbeing;
   wellbeing metrics to the organisation and the individual to monitor the individual's wellbeing;
   intervention triggers to the organisation to manage the individual's wellbeing.

### BRIEF DESCRIPTION OF DRAWINGS

Embodiments of the invention will now be described by way of example only with reference to the accompanying drawings, in which:

FIG. 1 is a block diagram of a system for monitoring and managing wellbeing of an individual within an organisation according to an embodiment of the present invention;

FIG. 2 is a flow diagram of a method performed by the system;

FIGS. 3 to 5 are example user interfaces of the system and method for receiving wellbeing data from the individual;

FIGS. 6 to 15 are example user interfaces for providing wellbeing recommendations to the individual;

FIG. 16 is an example decision tree for providing wellbeing recommendations to the individual;

FIGS. 17 and 18 are example wellbeing dashboards and wellbeing reports provided to the individual;

FIGS. 19 to 23 are example wellbeing dashboards and wellbeing reports provided to the organisation;

FIG. 24 is a diagram of an example machine learning model for providing intervention triggers to the organisation to support and manage the individual's wellbeing

FIGS. 25 to 32 are example user interfaces for the individual to submit help tickets or support requests to the organisation;

FIGS. 33 and 34 are example support dashboards and support reports for the organisation to manage help tickets or support requests of individuals; and

FIG. 35 is an example user interface provided to the organisation displaying resources and insights available to the organisation to support and manage the individual's wellbeing.

### DESCRIPTION OF EMBODIMENTS

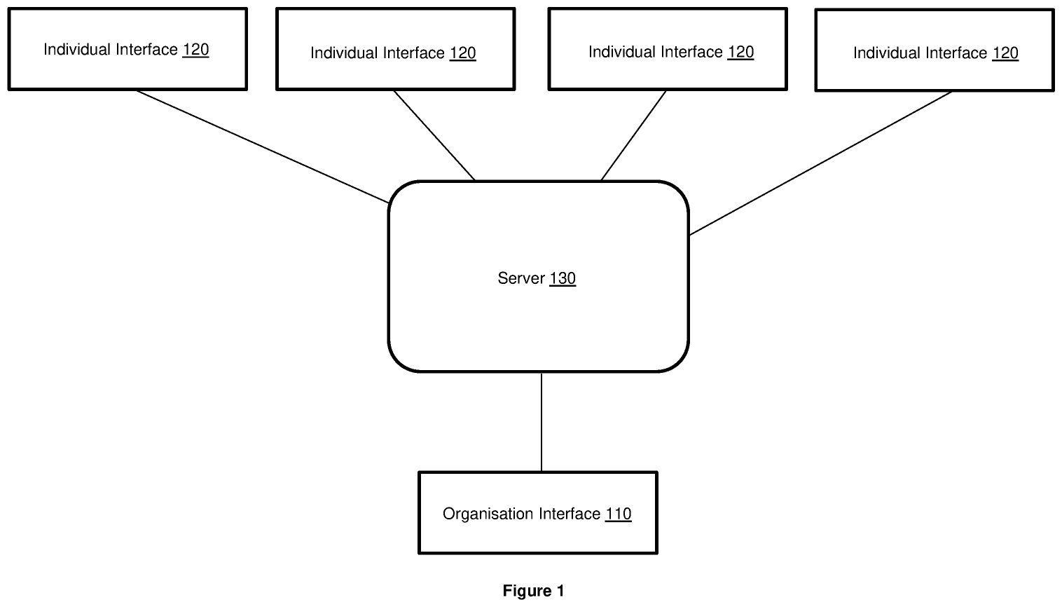

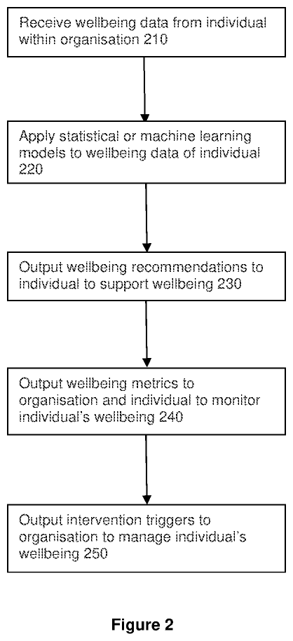

Referring to FIGS. 1 and 2, a system 100 for monitoring and managing wellbeing of an individual within an organisation according to an embodiment of the present invention may generally comprise an organisation interface 110 and individual interfaces 120 in wireless communication with a server 130. The organisation interface 110 may be associated with an organisation, such as an educational institution (eg, a school, a university, a college, etc), a business, a sports club, etc. The individual interfaces 120 may be associated with individuals within the organisation, such as students of the educational institution, employees of the business, athletes of the sports club, etc.

The organisation interface 110 and individual interfaces 120 may be implemented as web or mobile applications executable on mobile computing devices or desktop computers. The server 130 may be implemented as a cloud server using cloud processing and data storage systems. The server 130 may be configured to perform a method 200 to provide wellbeing monitoring and management services as Platform as a Service (PaaS) or Software as a Service (SaaS) to the organisation and individuals within the organisation.

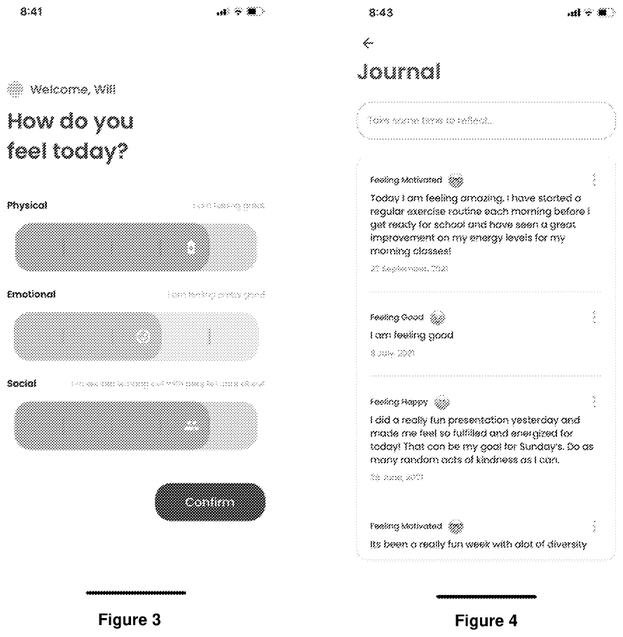

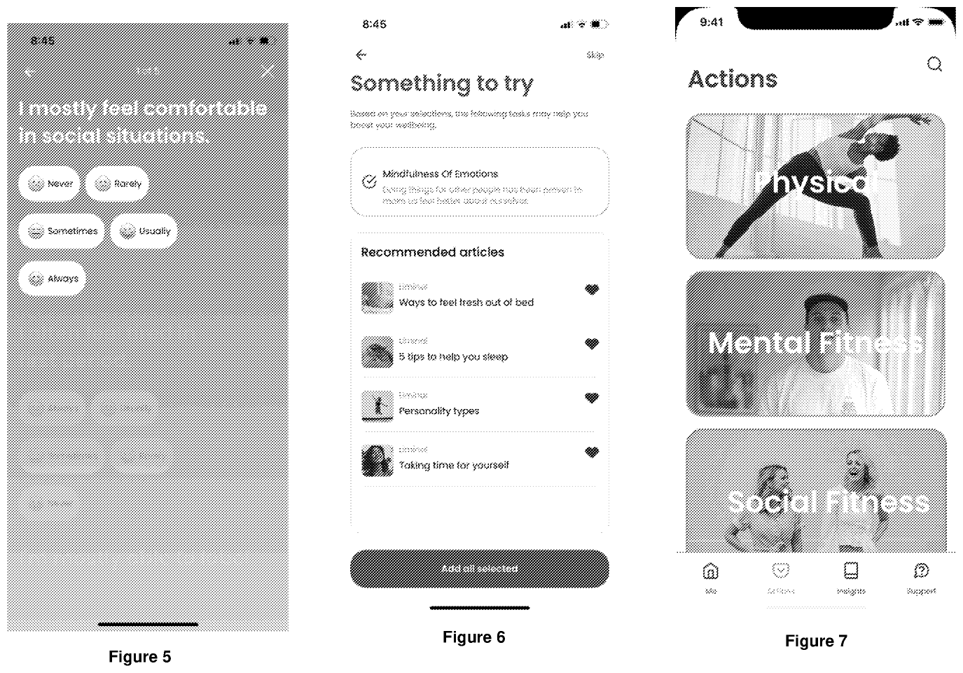

The method 200 may start at step 210 by receiving, via the individual interface 120, wellbeing data relating to an individual within the organisation. The wellbeing data may comprise data relating to physical, mental, emotional, and social wellbeing of the individual. The wellbeing data may, for example, comprise survey data, daily check-in data, and help-ticket data. For example, the survey data and daily check-in data may comprise data relating to relationships, sleep, mood, learning, and exercise of the individual. The wellbeing data may be self-recorded by the individual by user input comprising text, icons, slider bars, gestures, and scroll bars displayed in the individual interface. FIGS. 3 to 5 are example user interfaces provided via the individual interface 120 for receiving wellbeing data from the individual.

Next, at step 220, the server 130 may apply one or more statistical or machine learning models to the wellbeing data of the individual. The one or more statistical models may comprise descriptive statistics, cluster analysis, forecasting, survival analysis, log it model, or any combination thereof. The one or more statistical or machine learning models may comprise a neural network, a convolutional neural network, deep learning, decision tree learning, a random forest, association rule learning, inductive logic programming, support vector learning, a Bayesian network, a regression-based model, principal component analysis, or any combination thereof.

The one or more statistical or machine learning models may be pre-trained using wellbeing data of a plurality of individuals within a plurality of organisations. For example, labelled wellbeing data may be obtained from self-filled surveys, and one or more statistical or machine learning models may be built on a training data set using both supervised and unsupervised machine learning algorithms. The one or more statistical or machine learning models may comprise one or more black-box and white-box machine learning models.

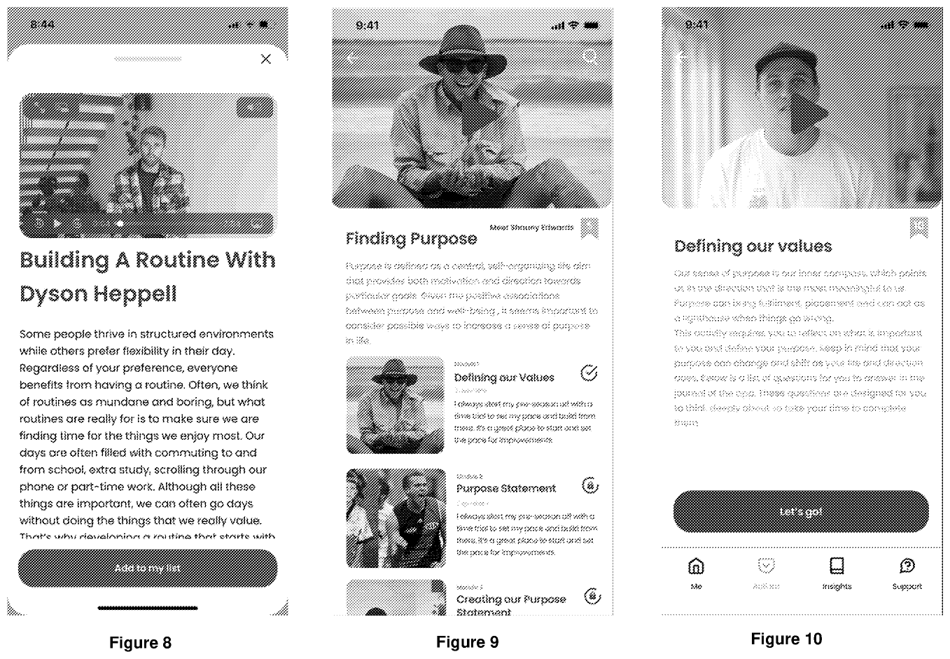

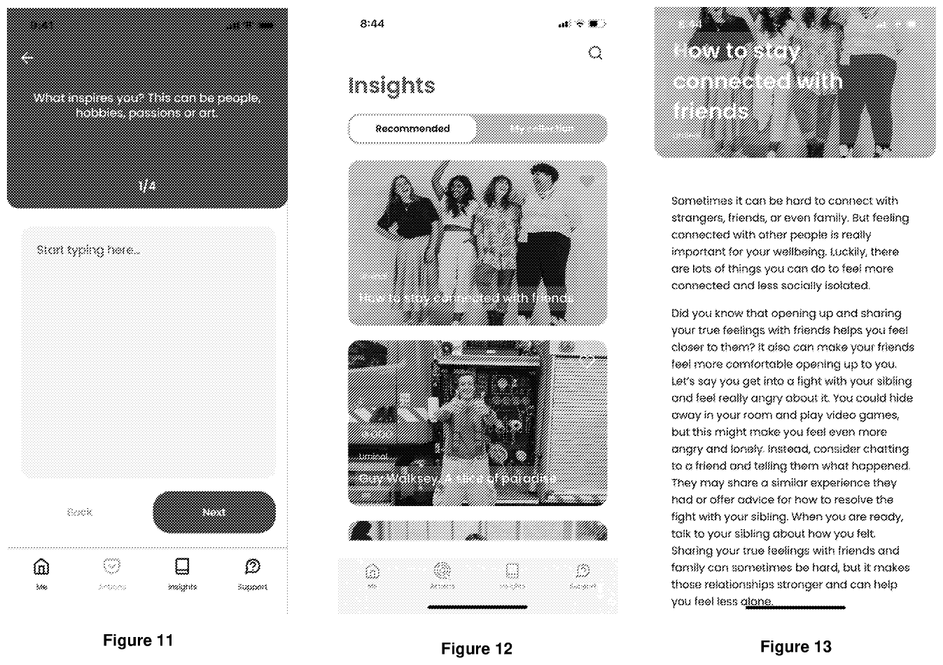

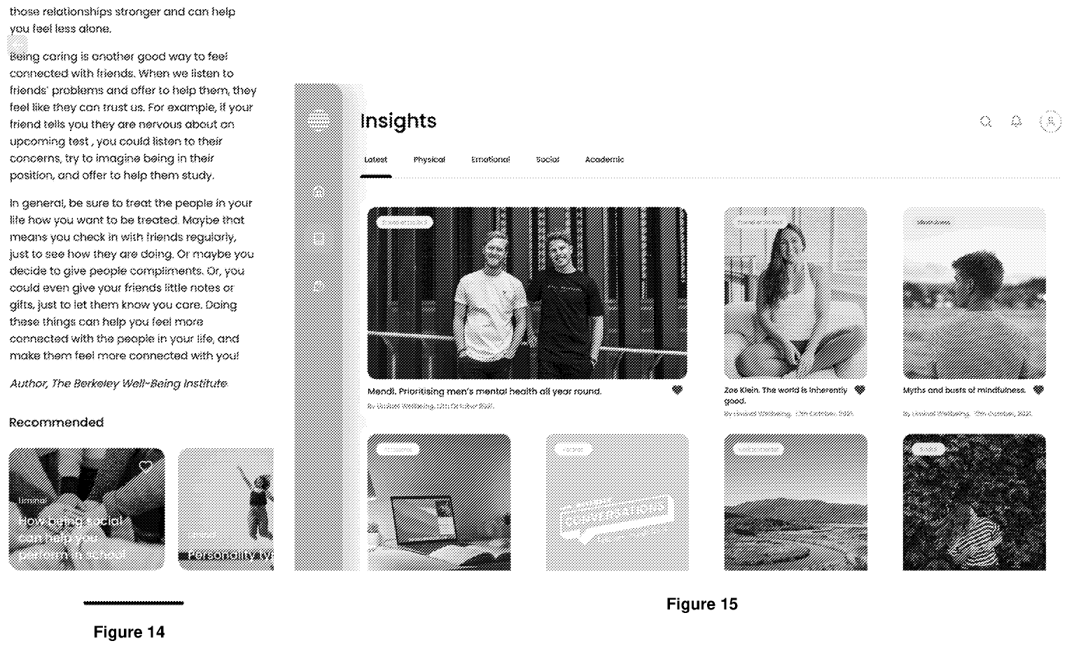

At step 230, one output of the one or more statistical or machine learning models may be wellbeing recommendations that are provided by the server 130 to the individual via the individual interface 120. The wellbeing recommendations provided to the individual may, for example, comprise targeted recommended actions and targeted recommended digital content to support their wellbeing. The wellbeing recommendations may further comprise insights, activities, feedback, tips, tools, content, journals, and support relating to wellbeing of the individual. FIGS. 6 to 15 are example user interfaces provided via the individual interface 120 for providing the wellbeing recommendations to the individual.

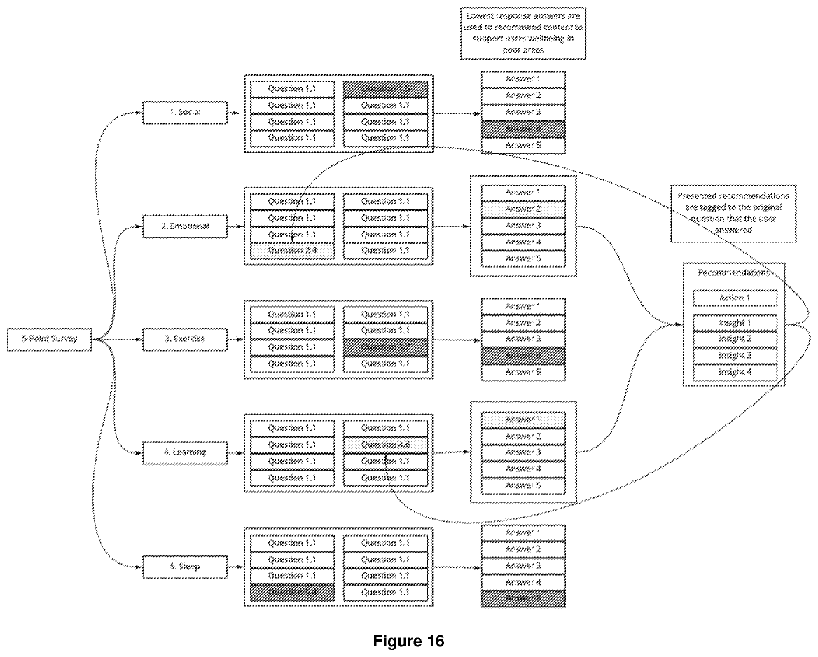

The wellbeing recommendations may be generated by applying the one or more statistical or machine learning models to the wellbeing data, for example, the survey data of the individual. FIG. 16 is a diagram of an example decision tree applied by the server 130 for providing wellbeing recommendations to the individual via the individual interface 120. The decision tree may be based on tagged answers provided by the individual to questions in a 5-point survey on topics comprising social, emotional, exercise, learning, and sleep.

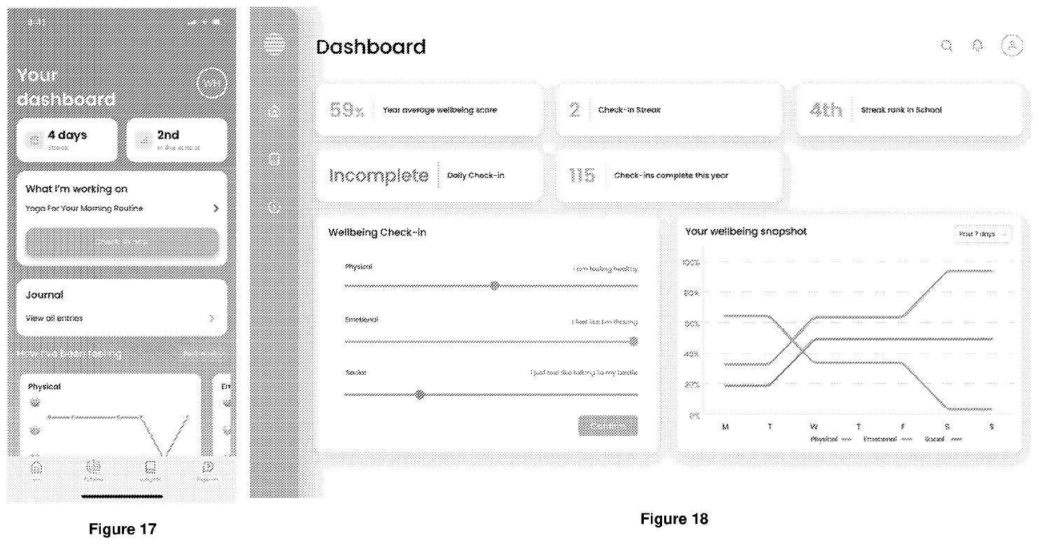

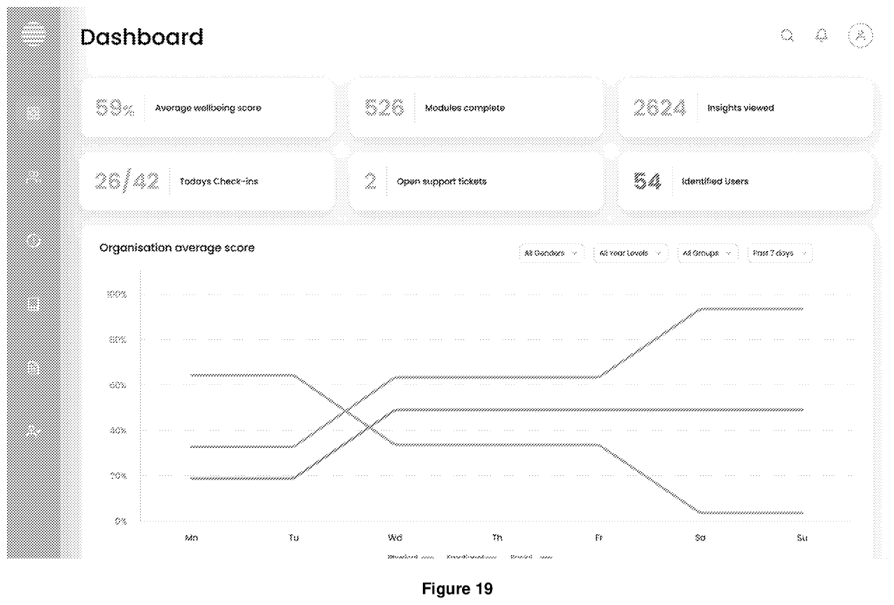

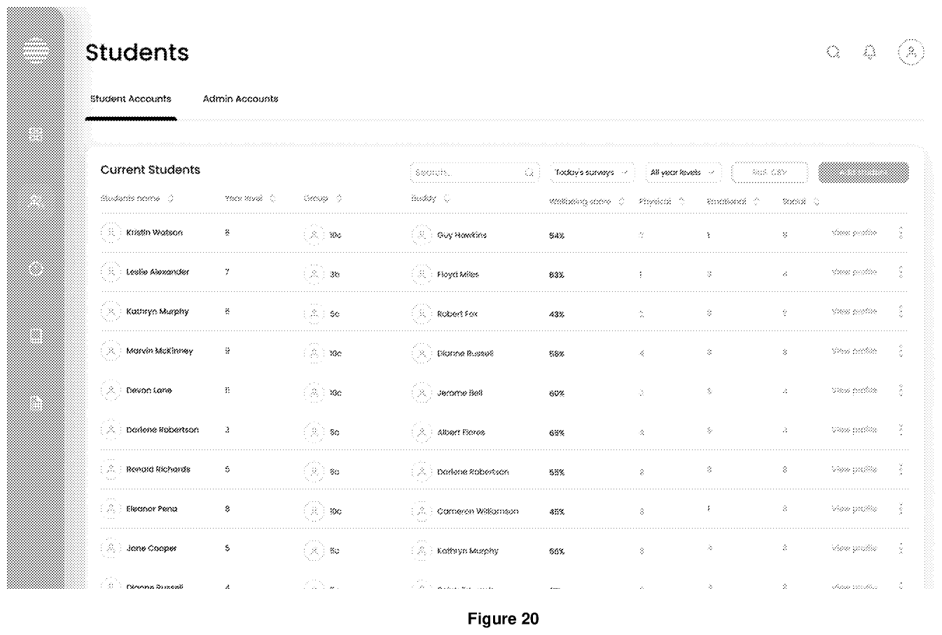

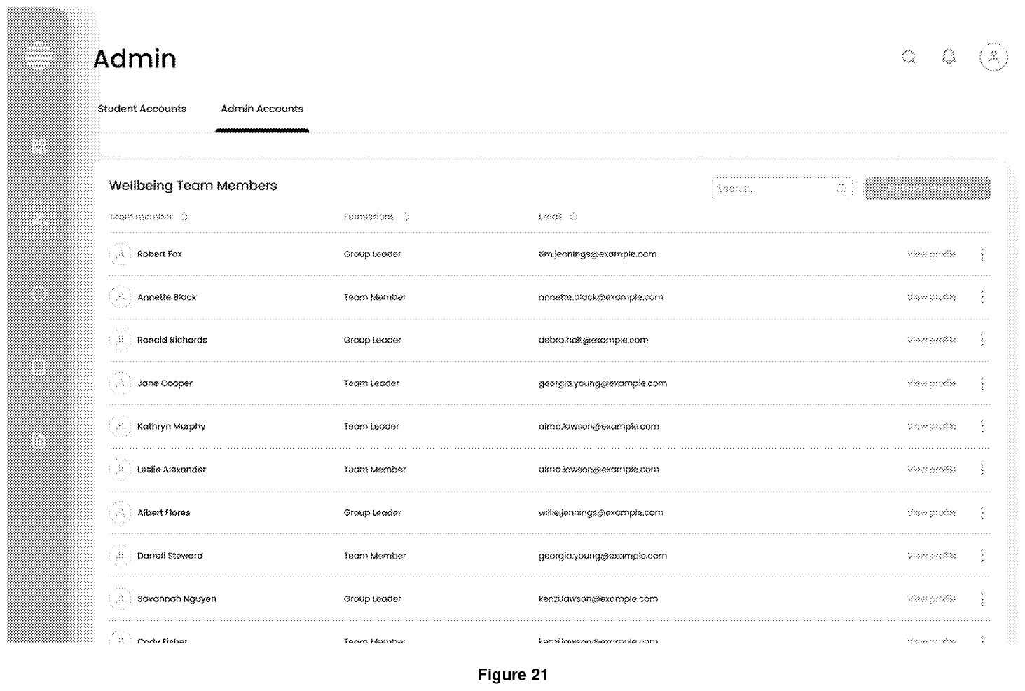

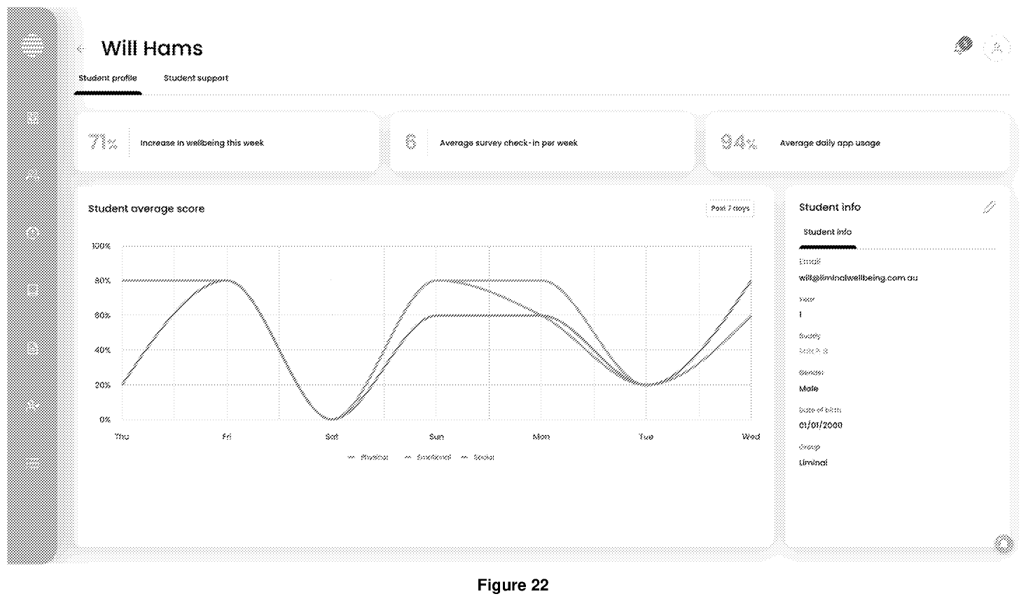

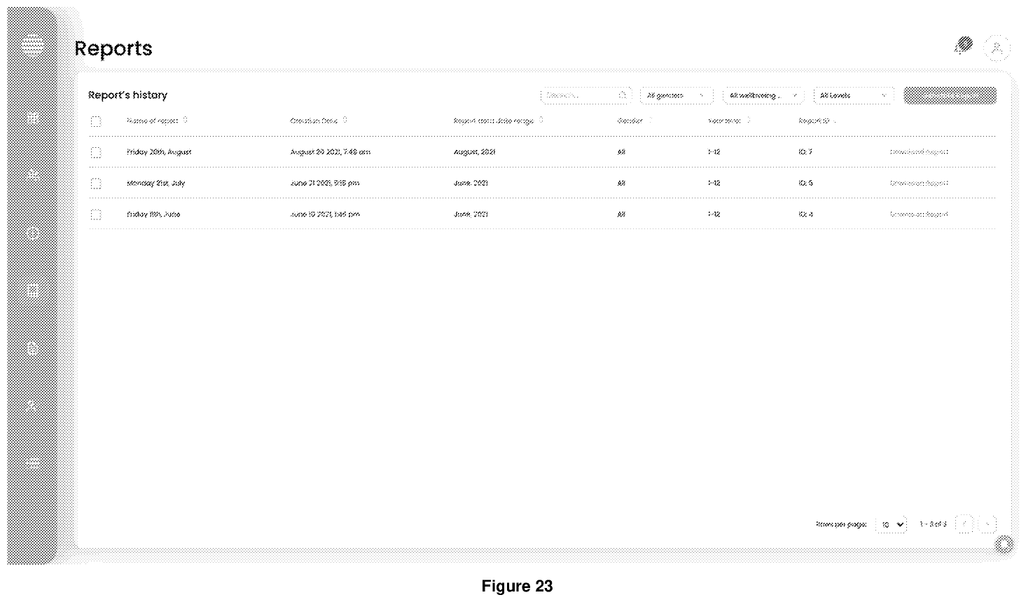

Next, at step 240, another output of the one or more statistical or machine learning models may be wellbeing metrics of the individual that are provided by the server 130 to the organisation via the organisation interface 110, and to the individual via the individual interface 120. The wellbeing metrics provided to the organisation and the individual may, for example, comprise wellbeing scores and wellbeing trends of the individual. The wellbeing scores and wellbeing trends of the individual may be provided to the organisation and the individual via wellbeing dashboards and wellbeing reports. FIGS. 17 and 18 are example wellbeing dashboards and wellbeing reports provided to the individual via the individual interface 120. FIGS. 19 to 23 are example wellbeing dashboards and wellbeing reports provided to the organisation via the organisation interface 110.

The wellbeing metrics of the individual may be generated by applying the one or more statistical or machine learning models to the wellbeing data, for example, the survey data and daily check-in data of the individual. For example, the wellbeing metrics may be calculated using the example statistical model algorithms below.

Wellbeing Score:

Sum of (physical_score/no. of days)+(Total Score physical(30)+social(30)+emotional(40)100
For each survey, the wellbeing core is calculated as:

physical_score=(30/5*survey score for physical+social_score=(30/5*survey score for social+emotional score=(40/5*survey score for emotional

Example: physical_score=(30/5*2 social_score=(30/5*1 emotional score=(40/5*3 12 8 24 44%

Average Wellbeing Score:

social_score/no. of days)+(emotional score/no. of days)/3

Average Survey Check-Ins Per Week:

total no. of surveys/no. of days.

At step 250, a further output of the one or more statistical or machine learning models may be intervention triggers that are provided by the server 130 via the organisation interface 110 to the organisation to manage the individual's wellbeing. The intervention triggers may be actioned by a wellbeing officer within the organisation, such as a student counsellor within an educational institution, a human resources officer within a business, or a coach or support staff within a sports club.

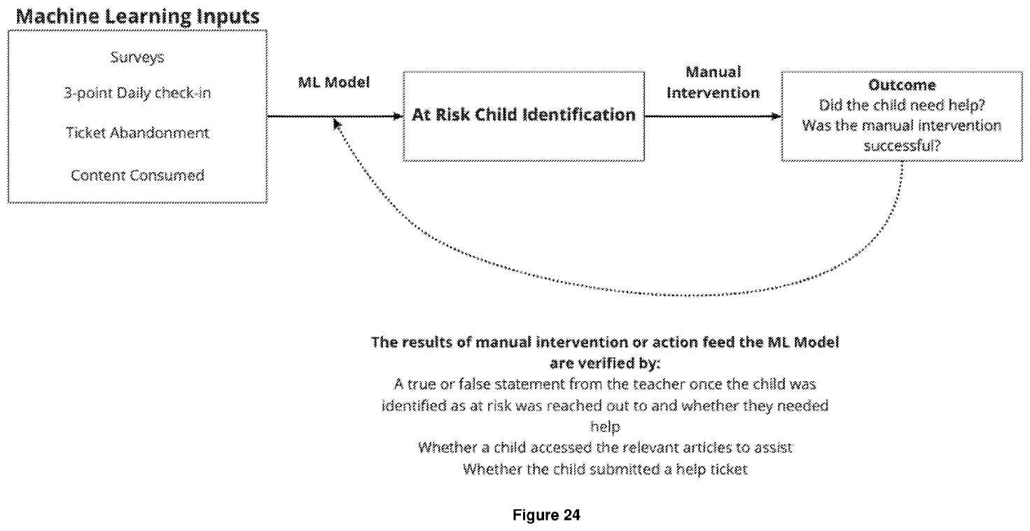

The intervention triggers may be generated by applying the one or more statistical or machine learning models to the wellbeing data. For example, the intervention triggers provided by the server 130 to the organisation via the organisation interface 110 may be generated by applying the one or more statistical or machine learning models to the survey data, daily check-in data, help-ticket data, and whether or not the individual has actioned the wellbeing recommendations. FIG. 24 is a diagram of an example machine learning model for determining if the individual is at risk to trigger intervention by the organisation to support and manage the individual's wellbeing.

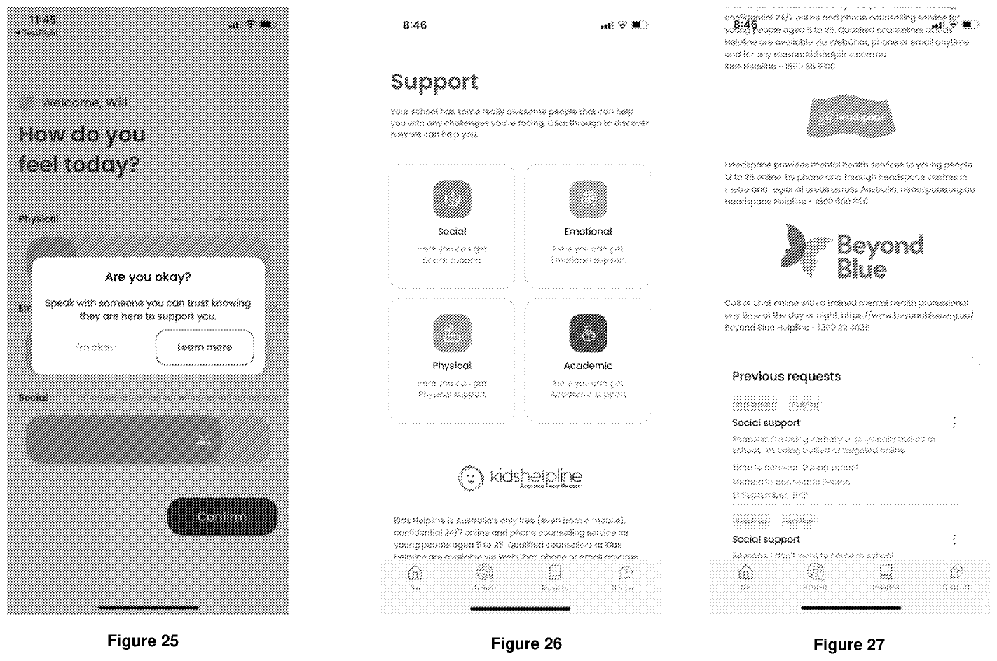

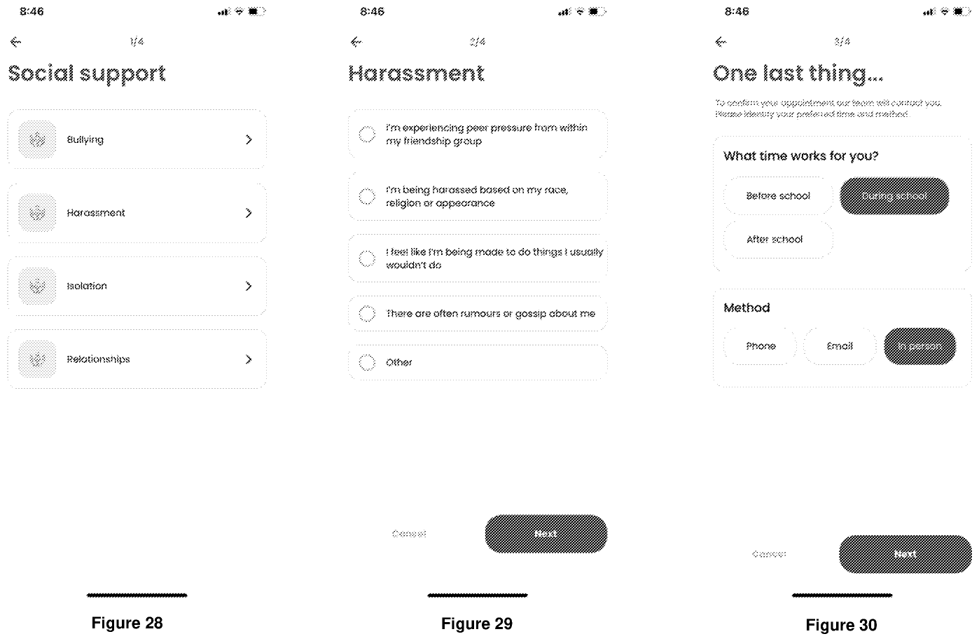

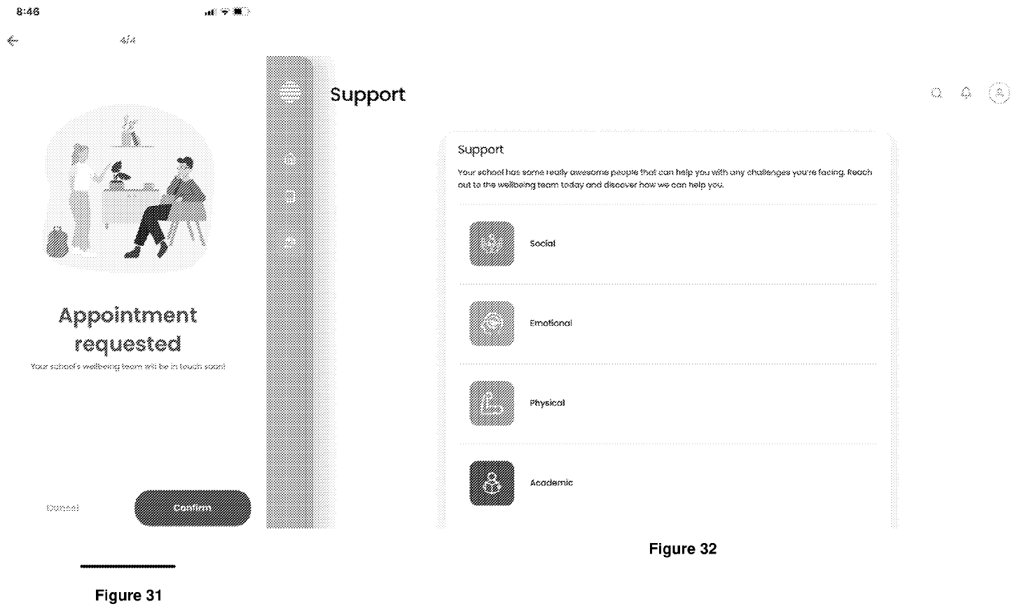

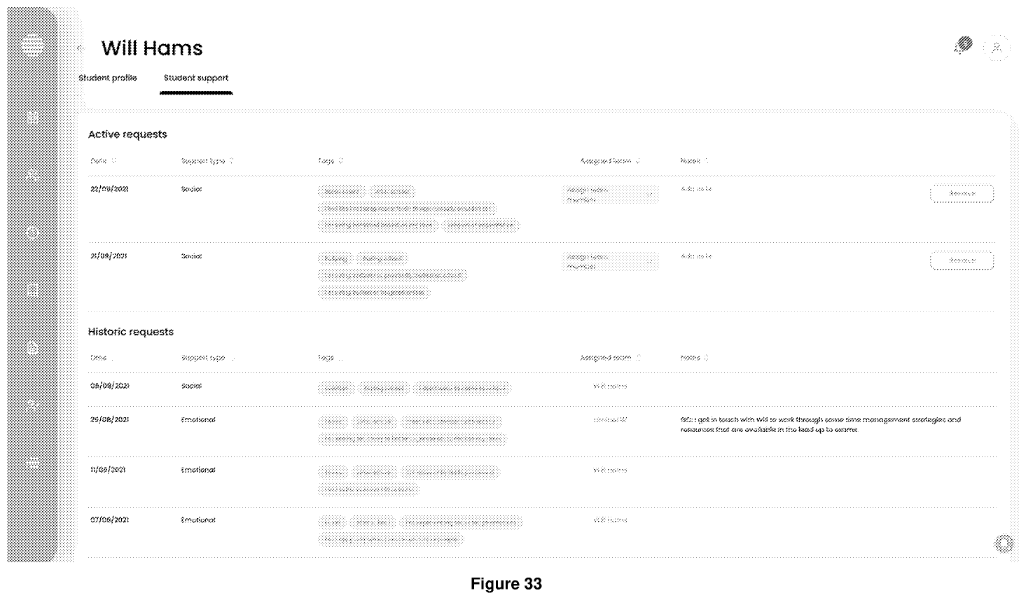

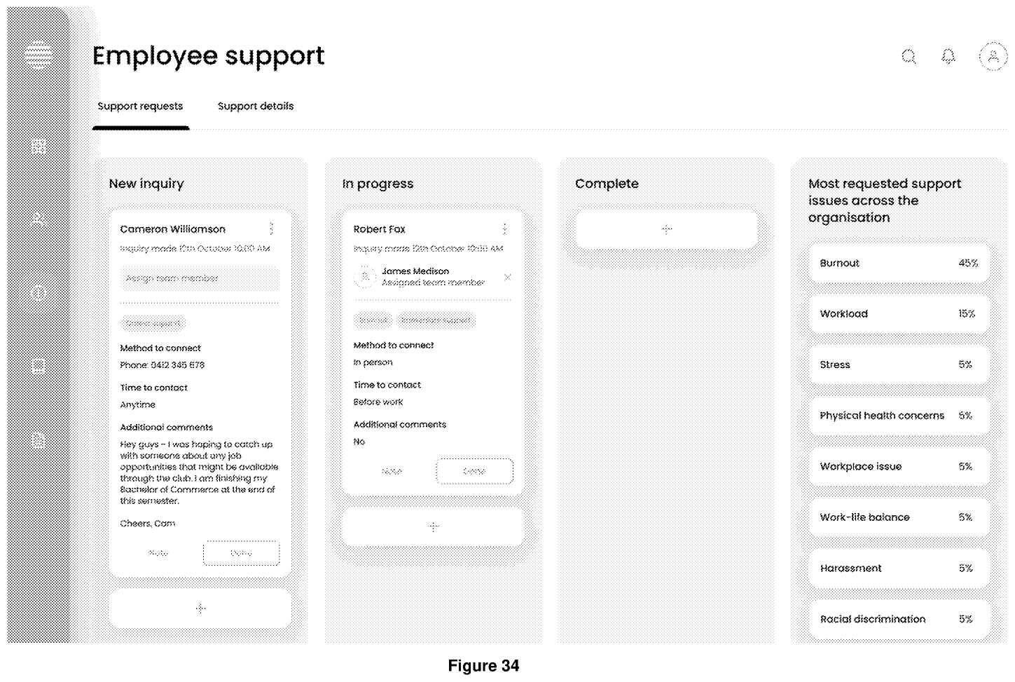

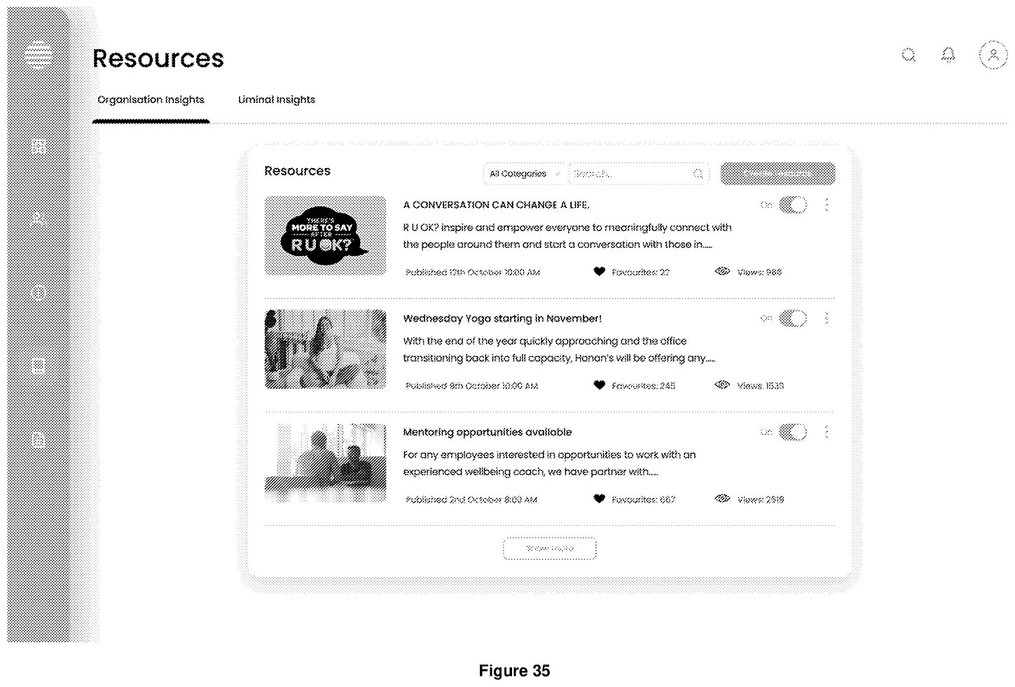

In addition to the intervention triggers provided to the organisation by the server 130, the individual may submit help tickets or requests for wellbeing support to the organisation via the individual interface 120 to trigger manual intervention by the organisation to support and manage the individual's wellbeing. FIGS. 25 to 32 are example user interfaces for the individual to submit help tickets or support requests to the organisation via the individual interface 120. The help tickets or support requests received from individuals within the organisation may be managed by the organisation via support dashboards and support reports. FIGS. 33 and 34 are example support dashboards and support reports provided to the organisation by the server 130 via the organisation interface 110. FIG. 35 is an example user interface provided by the server 130 to the organisation via the organisation interface 110 to display resources and insights available to the organisation to support and manage the individual's wellbeing.

Examples of user interfaces, dashboards and reports have been provided for examples of individuals and organisations, such as students of schools and employees of businesses. The invention is not limited to the examples that have just been given. In other words, those skilled in the art will appreciate that the examples may be reproduced for other types of organisations and individuals without difficulty, and with similar success, by substituting any of the generically or specifically described system components, method steps, and statistical or machine learning models mentioned anywhere in this specification for those actually used in the preceding examples.

Embodiments of the present invention provide a method and system that are both generally and specifically useful for applying one or more statistical or machine learning models to wellbeing data received from individuals within organisations to measure, support, monitor and manage their physical, social, mental and emotional wellbeing.

For the purpose of this specification, the word “comprising” means “including but not limited to,” and the word “comprises” has a corresponding meaning.

The above embodiments have been described by way of example only and modifications are possible within the scope of the claims that follow.

1. A system, comprising:
a memory; and
a processor configured by instructions stored in the memory to:
receive wellbeing data from an individual within an organisation;
apply one or more statistical or machine learning models to the wellbeing data of the individual to provide:
wellbeing recommendations to the individual to support their wellbeing;
wellbeing metrics to the organisation and the individual to monitor the individual's wellbeing;
intervention triggers to the organisation to manage the individual's wellbeing.

2. The system of claim 1, wherein the wellbeing data comprises survey data, daily check-in data, and help-ticket data.

3. The system of claim 2, wherein the wellbeing recommendations provided to the individual comprise recommended actions and recommended digital content to support their wellbeing.

4. The system of claim 2, wherein the wellbeing recommendations are generated by applying the one or more statistical or machine learning models to the survey data of the individual.

5. The system of claim 2, wherein the wellbeing metrics provided to the organisation and the individual comprise wellbeing scores and wellbeing trends of the individual and the organisation.

6. The system of claim 5, wherein the wellbeing scores and wellbeing trends of the individual and the organisation are generated by applying the one or more statistical or machine learning models to the survey data and daily check-in data of the individual.

7. The system of claim 5, wherein the wellbeing scores and wellbeing trends of the individual and the organisation are provided to the organisation and the individual via wellbeing dashboards and wellbeing reports.

8. The system of claim 2, wherein the intervention triggers provided to the organisation are generated by applying the one or more statistical or machine learning models to the survey data, daily check-in data, help-ticket data, and whether or not the individual has actioned the wellbeing recommendations.

9. A method, comprising:
receiving, at a processor, wellbeing data from an individual within an organisation;
applying, by the processor, one or more statistical or machine learning models to the wellbeing data of the individual to provide:
wellbeing recommendations to the individual to support their wellbeing;
wellbeing metrics to the organisation and the individual to monitor the individual's wellbeing;
intervention triggers to the organisation to manage the individual's wellbeing.

10. The method of claim 8, wherein the wellbeing data comprises survey data, daily check-in data, and help-ticket data.

11. The method of claim 10, wherein the wellbeing recommendations provided to the individual comprise recommended actions and recommended digital content to support their wellbeing.

12. The method of claim 10, wherein the wellbeing recommendations are generated by applying the one or more statistical or machine learning models to the survey data of the individual.

13. The method of claim 10, wherein the wellbeing metrics provided to the organisation and the individual comprise wellbeing scores and wellbeing trends of the individual and the organisation.

14. The method of claim 13, wherein the wellbeing scores and wellbeing trends of the individual and the organisation are generated by applying the one or more statistical or machine learning models to the survey data and daily check-in data of the individual.

15. The method of claim 13, wherein the wellbeing scores and wellbeing trends of the individual and the organisation are provided to the organisation and the individual via wellbeing dashboards and wellbeing reports.

16. The method of claim 10, wherein the intervention triggers provided to the organisation are generated by applying the one or more statistical or machine learning models to the survey data, daily check-in data, help-ticket data, and whether or not the individual has actioned the wellbeing recommendations.
